(19) United States
(12) Patent Application Publication
(10) Pub. No.: US 2011/0054195 A1
(43) Pub. Date: Mar. 3, 2011

(54) Synchronized strains of subepidermal cells of muscadine (muscadine sp.) grapevine pericarp for use as a sourse of flavonoids (nutraceuticals)

(76) Inventor: Violeta Colova, Tallahassee, FL (US)

(21) Appl. No.: 12/461,805

(22) Filed: Aug. 25, 2009

(51) Int. Cl. C07D 311/04; C12N 5/00; C12N 5/04

(52) U.S. Cl. 549/398; 435/420; 435/410

(57) ABSTRACT

Methods to generate and isolate novel Synchronized in vitro cell strains of Muscadinia sp. “Noble”var. and North American grape germplasm containing flavonoid compounds.

### BACKGROUND

1. Field

A method to generate and isolate novel Synchronized in vitro cell strains of Muscadinia sp “Nobel” var. and North American grape germplasm containing flavonoid compounds, and more particularly strains containing flavonoid (nutraceutical) compounds of “Muscadinia ”grape berries, and North American grape germplasm.

2. Background

Exceptionally powerful anti-oxidants recently identified and isolated in many fruits and vegetables promise to generate significant health benefits; particularly in the area of disease prevention. These benefits will far outweigh those traditionally accepted as being derived from intensive vitamin supplement regimes. In this connection, scientific evidence has shown the benefits of plant-based nutrition for reducing the risk of cancer and cardiovascular disease. For example, grape (Vitis vinifera L.) are well known to contain compounds such as resveratrol and additional phenolics responsible for the “French paradox”. In particular, muscadine grapes (Muscadinia rotundifolia) are known to contain elevated levels of total phenolics compared to the European grapes. Moreover, muscadines are the only Vitis source known to contain significant levels of ellagic acid a novel anti-oxidant and chemopreventive compounds. Cell suspensions originating from grape-berries were proven to be a reliable alternative source for these therapeutic compounds that are routinely produced in the berry. Some of these compounds accumulate only after elicitation treatments aiming to induce their biosynthesis.

The generation of red grape cell lines from Calli from grape cross sections, and skin cells and the establishment of liquid cultures therefrom is disclosed in WO 2006/090388, wherein liquid cultures were established on solid media developed on a homogenous cell suspension in the same media combinations but lacking a gelling agent. The addition of DDT or either ascorbic acid or citric acid improved growth and inhibited cell necrogenesis of the berry derived suspension culture. All explant types were successfully utilized for the establishment of liquid cultures, and the cultures were subcultured every 7-10 days to fresh growing media.

Nutrapharmaceuticals from polyphenol-containing fruit extracts are known for their anti-inflammatory effects; however, the use of fruit extracts (grape extracts) as a source of these compounds is limited because of their high sugar content. Similarly, the use of red wine as a source of these compounds is limited due to its high alcoholic content. Further, it has been shown that the therapeutic effect of wine and grapes is dependant on species, location, year (annual climate), processing etc. Therefore reliance on red wine or grapes as a source of these compounds does not lead to a homogeneous or consistent supply of these compounds. Moreover, fruits are often contaminated by residual fungicides, pathogens, pesticides and pollutants.

Further, still the benefit of gastrointestinal delivery of polyphenols from red wines and fruit extracts is limited by its bioavailability to target tissues and cells, due to differences in their bioavailability during passage through the intestines so no correlation can be drawn between the abundance of a certain polyphenol in a given food and its concentration as an active and beneficial compound in vivo.

Consequently, there is a need for natural (phyto) compositions that are better defined, consistent and highly bioavailable.

### SUMMARY

Embodiments disclosed herein address the above stated needs by obtaining a high level of flavonoids by:

sterilizing the berries of the harvested plant; making multiple insertions on each sterilized berry; transferring the berries to a test tube containing liquid culture media, vitamins, plant growth regulators, a carbohydrate source and a solidifying agent; isolating an immobilizing the berries; and resuspending the berries in a liquid culture media.

### DETAILED DESCRIPTION

The word “exemplary” is used herein to mean “serving as an example, instance, or illustration”. Any embodiment described herein as “exemplary” is not necessarily to be construed as preferred or advantageous over other embodiments. Sterilization of the initial explant.

### Example

### Sterilization of the Initial Explants:

Immature berries at “veraison” developmental stage of muscadinia “Noble” var. were harvested in the vineyard, carefully excised with attached small segment of the stem to preserve the physiological sterility of the berry intact, rinsed for ½ hour under running tap water and surfaced sterilized with 75% Ethanol and 5% Commercial bleach solution.

### Immobilization of the Initial Explant at In Vitro Conditions:

Under sterile conditions in Class II type biological safety cabinet (Fisher Hamilton “Safedire”) individual berries were transferred to the 50 ml Comig sterile tubes containing 35 ml culture media defined as Nitsh and Nitch, 1968 or Gamborg et al. 1968 salts and vitamins with at least 0.1 mg/l plant growth regulators (auxin- naphthaleneacetic acid(NAA) and/or cytokinin-benzyladenine (BA) or kinetin) and a carbohydrate source as sucrose at least 10 mg/L and a solidifying agent (agar) at least 2 g/L. For the purpose of the initiation of callusogenesis from the supepidermal layer of the pericarp, multiple insertions with sterile scalpel blade were made on the surface of each berry.

### Isolation and In Vitro Immobilization of Individual Subepidermal Cell Strains

The plants are observed weekly for callusogenesis under stereo microscope. Periodical berries with initial callusogenesis are dissected, the seeds are excised and the two segments are transferred at 15 ml disposable Petri plates containing 10 ml culture media defined as the one at step 2.

### Synchronization of the Individual Cell Strains in Suspension Culture.

Samples 1 or 2 g (fresh weight) of only pigmented callus were resuspended in 15 of the liquid culture media defined as the one in the second step of immobilization after 1 week only 15 ml suspensions were diluted by adding equal amount of the same fresh medium. At day 14, all suspensions were filtered (stainless steel sieve, 1 mm pore size) to discard the largest aggregates. After centrifugation at 1000× for 5 Min., the packed cell volume (PCV) was adjusted to 1% (w/v). At the end of each subculture, the fresh weight of the collected solid faction is recorded in order to check suspension growth rate.

While the invention has been described in connection with specific embodiments thereof, it will be understood that it is capable of further modifications. This application intended to cover any variations, uses, or adaptations following, in general, the principles of the invention and including such departures from the from the present disclosure within known or customary practice within the art to which the invention pertains and may be applied to the essential features hereinbefore set forth, and follows in the scope of the appended claims.

I claim:

1. An in vitro method of producing synchronized strains of subepidermal cells of muscadinia grape pericarp with high flavonoid content, comprising:
(A) sterilizing berries of the harvested excised plants;
(B) making multiple insertions on each sterilized berry, and transferring the berries to a test tube containing culture media, vitamins, plant growth regulators, a carbohydrate source and a solidifying agent;
(C) isolating and immobilizing the berries which contain callus production;
(D) resuspending the berries of Step (C) in a liquid culture media; and
(E) synchronizing individual cell strains in the suspension culture to obtain cell strains with high content flavonoids.

2. The method of claim 1 where the harvested excised explant is Muscadinia sp. “Noble”var. and North American grape germplasm.

3. The product obtained by the method recited in claim 1.

4. The product obtained by the method recited in claim 2.

5. The method of claim 1, wherein sterilizing is affected using a mixture of water, ethanol and a bleach solution.

6. The method of claim 1, wherein individual subepidermal cell strains of said berries are isolated and immobilized.

7. High flavanoid content synchronized individual cell strains of grapevine plants in a suspension culture.

8. The cell strains of claim 7, wherein said harvested excised plant is Muscadinia sp. “Noble” var. and North American grape germplasm.

9. A purified flavanoid extract of the synchronized individual cell strains of grapevine plants of the suspension culture of claim 7.

10. A purified flavanoid extract of claim 9 selected from the group consisting of anthocyanins and proanthocyanidins.
